(19) United States
(12) Patent Application Publication
(10) Pub. No.: US 2003/0195171 A1
(43) Pub. Date: Oct. 16, 2003

(54) Method for inhibiting bone resorption with an alendronate and vitamin D formulation

(76) Inventors: Anastasia G. Daifotis, Westfield, NJ (US); Sidney Mazel, Basking Ridge, NJ (US); John Yates, Lansdale, PA (US)

Correspondence Address: MERCK AND CO INC, P O BOX 2000, RAHWAY, NJ, 070650907

(21) Appl. No.: 10/404,161

(22) Filed: Apr. 1, 2003

Related U.S. Application Data
(60) Provisional application No. 60/370,529, filed on Apr. 5, 2002.
(60) Provisional application No. 60/370,501, filed on Apr. 5, 2002.
(60) Provisional application No. 60/370,336, filed on Apr. 5, 2002.
(60) Provisional application No. 60/398,434, filed on Jul. 25, 2002.

(51) Int. Cl. A61K 31/675; A61K 31/59; A61K 31/663

(52) U.S. Cl. 514/89; 514/102; 514/167; 514/91; 514/94

(57) ABSTRACT

Composition and method for preventing or treating abnormal bone resorption in mammals, the composition characterized as containing, a supplementary effective amount of a non-activated metabolite of vitamin D₂ and/or D₃ and a pharmaceutically effective amount of bisphosphonate to provide vitamin D nutrition during treatment to facilitate normal bone formation and mineralization, while minimizing the occurrence of or potential for the complications associated with vitamin D insufficiency, such as hypocalcemia and osteomalacia. The method of preventing or treating may be further characterized by concomitantly administering the components simultaneously or alternately at dosing intervals selected from once-weekly, twice-weekly, bi-weekly, monthly, and bi-monthly.

### FIELD OF THE INVENTION

The present invention relates to methods for inhibiting bone resorption in a mammal in need thereof by providing supplemental vitamin D nutrition to facilitate normal bone mineralization and formation while minimizing the occurrence of or potential for the complications associated with vitamin D insufficiency and the administration of bisphosphonate resulting from bone resorption. The method of the invention provides adequate vitamin D nutrition, while minimizing the occurrence of or potential for complications of hypocalcemia and osteomalacia associated with excessive amounts of activated vitamin D. The method may be characterized by orally administering, to a mammal, a pharmaceutical composition containing, in combination, a supplementary amount of a non-activated metabolite of vitamin D₂ and/or D₃, and a pharmaceutically effective amount of a bisphosphonate, as a unit dosage, according to a continuous schedule having a dosing interval of once-weekly, twice-weekly, biweekly, monthly, and bimonthly. The present invention also relates to pharmaceutical compositions containing various amounts of the combination of supplemental vitamin D nutrition and bisphosphonates, medicaments and kits useful for carrying out these methods.

### BACKGROUND OF THE INVENTION

A variety of disorders in humans and other mammals are associated with abnormal bone resorption. Such disorders include, but are not limited to, osteoporosis, Paget's disease, periprosthetic bone loss or osteolysis, metastatic bone disease, hypercalcemia of malignancy, and arthritides (including but not limited to osteoarthritis and rheumatoid arthritis).

One of the most common of these disorders is osteoporosis, which in its most frequent manifestation occurs in postmenopausal women. Osteoporosis is a systemic skeletal disease characterized by a low bone mass and microarchitectural deterioration of bone tissue, with a consequent increase in bone fragility and susceptibility to fracture. Because osteoporosis, as well as other disorders associated with bone loss, are chronic conditions, it is believed that appropriate therapy will generally require chronic treatment.

Multinucleated cells called osteoclasts are responsible for causing bone loss through a process known as bone resorption. It is well known that bisphosphonates are selective inhibitors of osteoclastic bone resorption, making these compounds important therapeutic agents in the treatment or prevention of a variety of generalized or localized bone disorders caused by or associated with abnormal bone resorption. See H. Fleisch, Bisphosphonates In Bone Disease, From The Laboratory To The Patient, 3rd Edition, Parthenon Publishing (1997), which is incorporated by reference herein in its entirety.

Bisphosphonates are known in the art to bond to hydroxyapatite in bone and to inhibit the bone resorptive activity of osteoclasts. See H. Fleisch, Bisphosphonates In Bone Disease, From The Laboratory To The Patient, 3rd Edition, Parthenon Publishing (1997). For example, bisphosphonates are known to be useful in the prevention of bone loss and in the treatment of such diseases as, but not limited to, osteoporosis, osteopenia, metastatic bone disease, multiple myeloma, periodontal disease, tooth loss, hyperparathyroidism, arthroses, Paget's disease, periprosthetic bone loss or osteolysis, and hypercalcemia of malignancy. All of these conditions are characterized by bone loss, resulting from a relative imbalance between bone resorption, i.e. bone breakdown, and bone formation.

At present, a great amount of pre-clinical and clinical data exists for the potent bisphosphonate compound alendronate. Evidence suggests that other bisphosphonates such as ibandronate, minodronate, pamidronate, risedronate and zoledronate have many properties in common with alendronate, including high potency as inhibitors of osteoclastic bone resorption. An older bisphosphonate compound, etidronate, also inhibits bone resorption. However, unlike the more potent bisphosphonates, etidronate impairs mineralization at doses used clinically, and may give rise to osteomalacia, a condition resulting in an undesirable decrease in bone mineralization. See Boyce, B. F., Fogelman, I., Ralston, S. et al. (1984) Lancet 1(8381), pp. 821-824 (1984), and Gibbs, C. J., Aaron, J. E.; Peacock, M. (1986) Br. Med. J. 292, pp. 1227-1229 (1986), both of which are incorporated by reference herein in their entirety.

Despite their therapeutic benefits, bisphosphonates are poorly absorbed from the gastrointestinal tract. See B.J. Gertz et al., Clinical Pharmacology of Alendronate Sodium, Osteoporosis Int., Suppl. 3: S13-16 (1993) and B.J. Gertz et al., Studies of the oral bioavailability of alendronate, Clinical Pharmacology & Therapeutics, vol. 58, number 3, pp. 288-298 (September 1995), which are incorporated by reference herein in their entirety. Intravenous administration has been used to overcome this bioavailability problem. However, intravenous administration is costly, inconvenient, and often unacceptable to patients, especially when the subject must be given an intravenous infusion lasting several hours on repeated occasions.

If oral administration of the bisphosphonate is desired, relatively high doses must be administered to compensate for the low bioavailability from the gastrointestinal tract. To maximize bioavailability, it is generally recommended that the subject take the bisphosphonate on an empty stomach and fast for at least 30 minutes afterwards. However, many subjects find the need for such fasting on a daily basis to be inconvenient.

Until recently, oral bisphosphonate therapies generally fell into two categories: (1) those therapies utilizing continuous daily treatment, and (2) those therapies utilizing a cyclic regimen of treatment and rest periods. The continuous daily treatment regimens normally involve the chronic administration of relatively low doses of the bisphosphonate compound, with the objective of delivering the desired cumulative therapeutic dose over the course of the treatment period. However, because bisphosphonates should be taken on an empty stomach followed by fasting and maintenance of an upright posture for at least 30 minutes, many subjects find daily dosing to be burdensome. These factors can therefore interfere with subject compliance, and in severe cases even require cessation of treatment. Cyclic treatment regimens were developed because some bisphosphonates, such as etidronate, when given daily for more than several days, have the disadvantage of actually causing a decline in bone mineralization, i.e. osteomalacia. U.S. Pat. No. 4,761,406, to Flora et al., issued Aug. 2, 1988, which is incorporated by reference herein in its entirety, describes a cyclic regimen developed in an attempt to minimize the decline in bone mineralization while still providing a therapeutic anti-resorptive effect. Generally, cyclic regimens are characterized as being intermittent, as opposed to continuous treatment regimens, and have both treatment periods during which the bisphosphonate is administered and non-treatment periods to permit the systemic level of the bisphosphonate to return to very low levels. However, the cyclic regimens, relative to continuous dosing, appear to result in a decreased therapeutic anti-resorptive efficacy. Data on risedronate suggests that cyclic dosing is actually less effective than continuous daily dosing for maximizing antiresorptive bone effects. See L. Mortensen, et al., Prevention Of Early Postmenopausal Bone Loss By Risedronate, Journal of Bone and Mineral Research, vol. 10, supp. 1, p. s140 (1995), which is incorporated by reference herein in its entirety. Furthermore, these cyclic regimens are cumbersome to administer and have the disadvantage of low subject compliance, and consequently compromised therapeutic efficacy. It is disclosed in U.S. Pat. No. 5,994,329, which is incorporated by reference herein in its entirety, that bisphosphonates, such as alendronate, can be administered at a relatively higher unit dosage according to a continuous schedule having a dosing interval of once-weekly dosing.

Bisphosphonate therapy has been associated with hypocalcemia. During treatment with bisphosphonates, the early inhibition of bone resorption induces a decrease in serum calcium, which occurs within days to weeks of the start of treatment. The serum calcium decrease can persist for many weeks to months following the initiation of treatment and can be prominent in vitamin D-insufficient patients. The hypocalcemic response can occasionally be severe enough to be symptomatic and warrant clinical intervention, particularly in patients with hypoparathyroidism and in cancer patients (See Vasikaran, S.D., Ed., Bisphosphonates: An Overview with Special Reference to Alendronate, Ann. Clin. Biochem. (2001)' 38: 608-623). As a result, adequate vitamin D and calcium intake is recommended for subjects using bisphosphonates. Vitamin D supplementation becomes even more critical when calcium needs are elevated due to the net influx of calcium into bone that occurs as a result of bisphosphonate therapy during effective osteoporosis treatment. Adequate vitamin D intake is essential to facilitate intestinal absorption of calcium, plays a critical role in regulating calcium metabolism, and is critically important in the mineralization of the skeleton. The primary biological function of vitamin D is to maintain calcium homeostasis by increasing the intestine's efficiency in absorbing dietary calcium and thereby helping ensure that the amount of calcium absorbed is adequate to maintain blood calcium in the normal range and adequate to maintain skeletal mineralization.

Vitamin D₂ (ergocalciferol) and vitamin D₃ (cholecalciferol) are non-activated forms of vitamin D. Vitamin D₃ is a precursor of the hydroxylated, biologically active metabolites and analogues of vitamin D₃, i.e. 1α-hydroxy-cholecalciferol, and 1α,25-dihydroxy-cholecalciferol. Generally cholecalciferol may be activated by hydroxylation into 25-hydroxy-cholecalciferol (a non-activated vitamin D₃ analogue), and 25-hydroxy-cholecalciferol may be further hydroxylated at the 1α-position to 1,25-dihydroxy-cholecalciferol (an active form of vitamin D₃). Vitamins D₂ and D₃ have similar biological efficacy in humans. Unlike 25-hydroxylated-vitamin D₃, a non-activated metabolite of vitamin D₃, “active vitamin D₃ analogs,” e.g. 1α-hydroxy-vitamin D₃ and 1α,25-dihydroxy-cholecalciferol, cannot be administered in large dosages on an intermittent schedule due to their toxicity to mammals. However, 25-hydroxy-cholecalciferol, a non-activated vitamin D₃ metabolite and the primary storage form of vitamin D in the human body, may be administered in larger doses on an intermittent basis than “active” forms of vitamin D without toxicity. The intrinsic activity of 25-hydroxy-cholecalciferol is about 100 fold lower than that of 1α,25-dihydroxy-cholecalciferol. The phrase “intrinsic activity” may be defined as the ability of the vitamin D analog to act as an agonist at the level of the vitamin D receptor, without need for enzymatic activation by the 1α-hydroxylase enzyme, to either calcitriol itself (the natural hormone metabolite of vitamin D₃, also known as 1α,25-dihydroxy-cholecalciferol) or a chemically similar analog, e.g. 1α-hydroxy-cholecalciferol or dihydrotachysterol₂ which also do not require 1α-hydroxylation for activity. All other forms of vitamin D that require 1α-hydroxylation are considered non-activated, e.g. 24,25-dihydroxy-cholecalciferol, vitamin D₂, vitamin D₃, and 25-hydroxy-cholecalciferol. See Philip Felig, M. D. et al., Endocrinology & Metabolism, 4^{th} Edition, McGraw-Hill, Inc., Medical Publishing Division, pp. 1098-1109 (2001), which is incorporated herein in its entirety by reference thereto.

Vitamin D insufficiency is recognized as causes of metabolic bone disease in adults. Vitamin D insufficiency is characterized by the impairment of calcium and phosphate absorption but normal bone mineralization and a serum 25-hydroxy vitamin D level between about 9 to about 15 ng/mL. Vitamin D deficiency is characterized by impaired bone mineralization due to a serum 25-hydroxy vitamin D level of <about 9 ng/mL. Vitamin D insufficiency and deficiency result in increased parathyroid hormone (PTH), which in turn causes increased osteoclastic activity and calcium loss from bone, which in turn aggravates osteoporosis, especially in older adults. Sustained vitamin D insufficiency and deficiency are thought to be an important cause of gradual bone loss. Depending on the degree of the vitamin D and calcium deficiency, the histological picture may either be one of osteomalacia or osteoporosis.

Vitamin D insufficiency can be age related, or due to geographical and seasonal causes. While exposure to sunlight provides most of the vitamin D required for children and young adults, the body can deplete its stored vitamin D because of a lack of exposure to sunlight combined with a dietary deficiency. Darkly pigmented skin and the skin of the elderly are believed to be less efficient in synthesizing vitamin D₃, especially during the winter months and in northern latitudes. Aging and renal impairment can also reduce the efficiency of vitamin D metabolism. To further compound this problem, through an independent mechanism, the efficiency of intestinal calcium absorption decreases with increasing age. Although vitamin D₃ can be derived from dietary sources, the amounts of constitutive vitamin D₃ in foods is low. To compensate for dietary deficiencies, some countries supplement certain foods, such as milk, margarine, cereals, and bread with vitamin D (Glenville, J., Pharmacological Mechanisms of Therapeutics: Vitamin D and Analogs, Principles of Bone Biology, 1069-1081 (1996)). However, vitamin D supplementation of food fails to ensure adequate intake, especially among the elderly who do not frequently consume these foods. As a result, vitamin D deficiency is particularly problematic in older people where intestinal absorption of calcium is less efficient, and dietary deficiencies and low sunlight exposure are common.

Vitamin D deficiency and vitamin D insufficiency remain neglected problems. In New England during the winter, it is estimated that 57% or more of inpatients and 40% of outpatients are vitamin D insufficient or deficient (Malabanan, A. et al., Redefining Vitamin D deficiency. Lancet 351, 805-806 (1998)). Approximately 30% of osteoporotic patients in the United States, European Union and Asia have some degree of vitamin D insufficiency which may be reversed with vitamin D supplementation. The prevalence of low 25-hydroxy vitamin D₃ metabolite levels in elderly long-term care patients approaches 100% in Northern Europe and in North America. The prevalence of 25-hydroxy vitamin D₃ insufficiency and deficiency in healthy elderly in Northern Europe is about 50% and 15%, respectively. In North America and Scandinavia, nearly 25% of the elderly women have winter 25-hydroxy vitamin D₃ levels that are below normal limits. Finally, according to studies conducted in Europe, the majority of elderly patients with hip fractures had 25-hydroxy vitamin D levels within the osteomalacia range. Two-thirds of hip fracture patients in Northern Europe have vitamin D₃ deficiency. The prevalence of vitamin D insufficiency and deficiency creates a medical need for vitamin D supplementation in the patient populations prone to, or suffering from, osteoporosis or osteopenia and in the subjects undergoing bisphosphonate therapy.

In subjects undergoing bisphosphonate therapy, and in particular those subjects with inadequate dietary calcium intake or inadequate calcium absorption, there is a need for supplemental D nutrition to facilitate bone formation and mineralization, as well as prevent hypocalcemia by minimizing the potential for or occurrence of vitamin D insufficiency. A single product or preparation comprised of metabolites of vitamin D₂ and/or vitamin D₃ and a bisphosphonate would address this need by ensuring that patients receiving bisphosphonate also receive sufficient vitamin D.

Vitamin D supplementation is routinely used in clinical trials of bone resorption compounds and recommended on product labels and in product package circulars. However, in practice many patients fail to take the needed additional vitamin D supplements. Also, physicians may overlook this standard of care and/or patients may fail to comply with vitamin D nutritional supplement recommendations. Approximately 30% of the osteoporotic patients in the United States have some degree of vitamin D insufficiency.

There is also need for a bisphosphonate and non-activated metabolite of vitamin D₂ and/or vitamin D₃ combination product to enhance the overall efficacy of bisphosphonate treatment by supplementing vitamin D nutrition to facilitate calcium absorption. It is known and documented in the literature that bisphosphonates are poorly absorbed from the gastrointestinal tract (<1%) and that this limited absorption is further compromised by the presence of food and beverages other than water, which bind to bisphosphonates and greatly reduce their bioavailability (See Bone, H.G., Adami, S., Rizzoli, R. et al.; Weekly Administration of Alendronate; rationale and plan for clinical evaluation. Clin. Ther. 22:15-28 (2000)). There is evidence that food and beverages, including tea, coffee, and mineral water, can reduce the bioavailability of bisphosphonates. There is further evidence that calcium and magnesium containing products, such as calcium supplements, dairy products, antacids, and some oral medications, can chelate bisphosphonates and reduce or almost entirely prevent their absorption into the body. To ensure that bisphosphonate bioavailability is not further reduced, subjects are advised to take bisphosphonates on an empty stomach with plain water at least one-half hour before the first food, beverage, or medication of the day. This includes a restriction on taking antacids, calcium supplements and vitamins when taking bisphosphonates (see Physician's Desk Reference, Patient Information about Fosamax®, p. 2101, 5^{th} Edition, 2002). Patients must currently wait at least 30 minutes after taking alendronate before taking any vitamin D supplements or food supplemented with vitamin D.

Currently, patients taking oral bisphosphonates while requiring vitamin D supplementation are advised to take two separate products at two different times. Vitamin D supplements are most commonly taken daily, while bisphosphonates may be administered daily, weekly or at cyclical intervals. As a result, many patients being treated for osteoporosis or osteopenia fail to take vitamin D despite being advised to do so. Although patients can currently take vitamin D before or after taking their bisphosphonate dosages, there is evidence that patients do not. A 1998 marketing study showed that while 75-85% of physicians prescribing alendronate also recommended vitamin D supplementation, only 57% of osteoporotic patients actually complied.

Although vitamin D can also be supplemented in the form of a multivitamin, in the US many over-the-counter oral vitamin D supplements are not sold in the dosage units required for dosing less frequently than daily. There is currently no appropriate vitamin D product available for patients to use on a once-weekly, twice-weekly, bi-weekly, monthly, and bimonthly basis. Even a patient, self-administer vitamin D simultaneously or alternately with bisphosphonate dosage, it is possible that the vitamin D supplement could interfere with and reduce the bisphosphonate absorption.

Therefore, if the bioavailability issues can be overcome, it would be beneficial to combine vitamin D and a bisphosphonate in a single composition or formulation to administer to patients undergoing bisphosphonate therapy to facilitate normal bone formation and mineralization.

Prior to the present invention, there have been no clinical studies in which a bisphosphonate and a non-activated metabolite of vitamin D₂ and/or vitamin D₃ have been simultaneously administered in the same dosage unit. Furthermore, there have been no clinical studies in which a bisphosphonate and a non-activated metabolite of vitamin D₂ and/or D₃ were both administered weekly or monthly, alternately or simultaneously. While there is extensive data on the efficacy of alendronate in patients that received a separate vitamin D supplement, there is no human or preclinical data on the efficacy and bioavailability of alendronate, when alendronate and a non-activated metabolite of vitamin D₂ and/or D₃ are administered in a single formulation.

Bisphosphonates can be given in the same formulation with a non-activated metabolite of vitamin D₂ and/or D₃ without adversely affecting their bioavailability. Furthermore, it is believed that higher doses of vitamin D₂ and/or D₃ can be administered in the same composition with bisphosphonates without affecting their bioavailability if vitamin D₂ and/or D₃ is administered as non-active metabolites. It is known that vitamin D₂ can be used in place of vitamin D₃ with similar results as those found for vitamin D₃. Thus, administration of non-active metabolites of vitamin D₂ and/or D₃ in the same formulation with bisphosphonate eliminates the separate dosing requirements of vitamin D supplements during bisphosphonate treatment and allows vitamin D supplementation without adversely affecting the bioavailability and efficacy of bisphosphonate.

Patients would benefit from pharmaceutical composition containing a combination of non-activated metabolites of vitamin D₂ and/or D₃, and bisphosphonate because the composition would supply high doses of vitamin D nutrition to facilitate normal bone formation and mineralization, as well as enhance the efficacy of bisphosphonate treatment. From a patient lifestyle standpoint, the methods of the present invention would also be more convenient than daily or cyclic dosing regimens for bisphosphonates with additional daily vitamin D supplementation. It is believed that a non-activated metabolite of vitamin D₂ and/or D₃ may be given at monthly or longer intervals due to their long body half-life. As a result of this invention, patients will no longer need to take vitamin D daily to benefit from vitamin D supplementation, since this invention provides for once-weekly, twice-weekly, bi-weekly, once-monthly, and bi-monthly dosing of a non-activated metabolite of vitamin D₂ and/or D₃. Generally, patients will not need to keep track of a complex dosing regimen of separate bisphosphonate and vitamin D administration, and patients will be less frequently subjected to the inconvenience of taking the drug on an empty stomach and fasting for at least 30 minutes before or after dosing. The compositions and methods of the present invention are likely to have the advantage of promoting better patient compliance, which in turn can translate into better therapeutic efficacy.

The literature contains several patents that have disclosed combinations of active vitamin D₂ and/or D₃ metabolites in combination with bisphosphonates. However, the patent literature does not provide a pharmaceutical composition containing the combination of bisphosphonate and a non-activated metabolite of vitamin D₂ and/or D₃, characterized by a single product for oral dosing at intervals less frequent than daily or intervals more frequent than every 6 months or longer. U.S. Pat. No. 4,230,700, issued Oct. 28, 1980 discloses the conjoint daily administration of polyphosphonate compounds and vitamin D-like anti-rachitic compounds for inhibiting mobilization of calcium phosphates in animal issue. U.S. Pat. No. 4,330,537, issued May 18, 1982 discloses compositions used in the methods of U.S. Pat. No. 4,230,700. The polyphosphonate compounds claimed in these patents include non-amine containing bisphosphonates, as well as amino-, alkylamino- and dialkylamino-substituted bisphosphonates. International Patent Publication No. WO 90/01321 discloses pharmaceutical compositions containing a bisphosphonate and an activated vitamin D compound for daily oral dosing.

International Patent Publication No. WO 01/97788 discloses fixed combinations of bisphosphonates including vitamin D for treating conditions of abnormally increased bone turnover by intermittent administration, wherein the period between administrations is at least about 6 months, 1 year, 18 months, 2 years or less frequently. Several patents, including EP 0 381 296, EP 0 162 510 and U.S. Pat. No. 4,812,304, claim kits that provide for the cyclic administration of a bone activating compound, such as an active vitamin D metabolite, for 1-5 days; followed by administration of a bone resorption compound, such as alendronate, etidronate or another polyphosphonate, for 10-20 days; followed by a rest period during which either calcium or vitamin D is administered for 30-100 days or during which no supplements are administered for an interval of 70-180 days. The active vitamin D metabolites utilized in these patents are hydroxylated vitamin D₂ and D₃ metabolites, such as the 1α-hydroxy vitamin D₃ and 1,25-dihydroxy vitamin D₃ metabolites, which may be toxic if administered in large quantities. European Patent Publication No. EP 1 051 976 discloses the use of a preparation of bisphosphonate, or a mixture of bisphosphonates, and some combinations formed by pharmaceutically acceptable calcium salts, fluor salts, vitamin D, PTH, fractions of PTH and other hormones to treat osteogenesis imperfecta, however, the preparation is used in a repetitive cyclic treatment regimen with two treatment stages.

Several other patents claim combinations of active metabolites of vitamin D₃ and alendronate. Both International Publication No. WO 01/28564 Al and Japanese Patent Publication No. 7-330613 disclose compositions containing alendronate and activated forms of vitamin D₃, such as 1α-hydroxy or 1α,25-dihydroxy vitamin D₃. Additionally, Japanese Patent Publication No. 11-60489 discloses an osteoporosis preventative containing an activated form of vitamin D₃ and a bisphosphonate, wherein the activated forms of vitamin D₃ are hydroxylated at the 1α; 1α,24; 1α,25; and 1α,24,25 positions. International Publication No. WO 92/21355 discloses method of administering calcium, vitamin D, and a bisphosphonate on a daily basis.

International Publication No. WO 01/28564 A1 disclose an oral composition and method for the treatment of metabolic bone disease characterized as containing alendronate and calcitriol, an active vitamin D₃ derivative expressed as 1,25-dihydroxy-vitamin D₃, wherein the composition contains from 1,000 to 5,000 parts by weight of alendronate per one part by weight of calcitriol.

These patents do not provide for methods of preventing or treating metabolic bone disease, characterized by administering, to a mammal in need thereof, a composition containing a non-activated metabolite of vitamin D₂ and/or D₃, in combination with a bisphosphonate that is useful for continuous oral administration at dosing intervals less frequent than daily dosing and more frequent than dosing at 6 month or longer intervals, such as once-weekly or once-monthly dosing.

As a result, there is a need for a composition containing a non-activated metabolite of vitamin D₂ and/or D₃ and bisphosphonate to provide supplemental vitamin D to facilitate normal bone formation and mineralization, while minimizing the potential for, or occurrence of, complications associated with vitamin D insufficiency, such as hypocalcemia and osteomalacia. There is a need for a bisphosphonate and non-activated metabolite of vitamin D₂ and/or D₃ to provide a supplemental amount of vitamin D nutrition to facilitate normal bone formation and mineralization in subjects undergoing bisphosphonate therapy.

Prior to the present invention, there have been no clinical studies in which a bisphosphonate and a non-activated metabolite of vitamin D₂ and/or D₃ have been simultaneously administered in the same dosage unit. Furthermore, there have been no clinical studies in which a bisphosphonate and a non-activated metabolite of vitamin D₂ and/or D₃ were both administered weekly or monthly, alternately or simultaneously. While there is extensive data on the efficacy of alendronate where patients received a separate vitamin D supplement, there is no human or preclinical data on the efficacy and bioavailability of alendronate, when alendronate and a non-activated metabolite of vitamin D₂ and/or D₃ are administered in a single formulation.

It has been surprisingly found that a non-activated metabolite of vitamin D₂ and/or D₃ (given in doses up to 7 times higher than that activated metabolites of vitamin D₂ and/or D₃) can be simultaneously and alternately co-administered with a bisphosphonate, e.g. alendronate, without adversely effecting the bioavailability of the bisphosphonate.

### SUMMARY OF THE INVENTION

The present invention relates to a pharmaceutical composition, characterized as containing a non-activated metabolite of vitamin D₂ and/or D₃, and at least one bisphosphonate. The composition is suitable for inhibiting abnormal bone resorption in a mammal, in need thereof, when it provides a supplementary effective amount of non-activated metabolite of vitamin D₂ and/or D₃, a pharmaceutically effective amount of at least one bisphosphonate.

The invention also relates to a method for inhibiting bone resorption in a mammal, in need thereof, characterized by orally administering thereto a composition containing a supplementary effective amount of a non-activated metabolite of vitamin D₂ and/or D₃, and a pharmaceutically effective amount of at least one bisphosphonate. The composition may be administered as a unit dosage according to a continuous schedule having a dosing interval of once-weekly, twice-weekly, bi-weekly, once-monthly and bimonthly.

Similarly, the invention relates to methods of concomitantly administering the components of the composition, i.e. a non-activated metabolite of vitamin D₂ and/or D₃, and bisphosphonate simultaneously and alternately.

These and other embodiments of the invention, as will become apparent to those skilled in the art, are provided in the following detailed description.

### DETAILED DESCRIPTION OF THE INVENTION

The present invention relates to a method for inhibiting bone resorption in a mammal, in need thereof, by administering a composition containing a supplementary effective amount of a non-activated metabolite of vitamin D₂ and/or D₃ and a pharmaceutically effective amount of at least one bisphosphonate. The method has the advantage of facilitating normal bone mineralization and formation while minimizing the occurrence of or potential for the complications of vitamin D insufficiency during bisphosphonate therapy. The present invention also relates to methods, preferably oral methods, for inhibiting bone resorption in a mammal in need thereof by facilitating adequate vitamin D nutrition while minimizing the occurrence of or potential for the complications of hypocalcemia and osteomalacia. The present invention also relates to a method of treating or preventing abnormal bone resorption by providing a composition, characterized as containing a supplementary effective amount of a non-activated metabolite of vitamin D₂ and/or D₃ nutrition and a pharmaceutically effective amount of bisphosphonate. Alternatively, the method of treating or preventing abnormal bone resorption relates to concomitantly administering the bisphosphonate and metabolite of vitamin D, alternately or simultaneously, as separate, unit dosage compositions.

In the present method of the invention, it has been surprisingly found that a non-activated metabolite of vitamin D₂ and/or D₃, administered in doses up to 7 times higher that that administered on a daily basis, can be simultaneously or alternately administered with a bisphosphonate, such as alendronate, without adversely affecting the bioavailability of the bisphosphonate. Based on this study, oral administration of activated metabolites of vitamin D₃ together with an oral dose of alendronate has minimal to no effect on the bioavailability of alendronate. As a result, the invention may be effective to treat all of the indications for which alendronate is effective. The invention may be used to prevent bone loss, increase bone mass and treat any type of osteoporosis, osteopenia, or other bone diseases, including, but not limited to Paget's disease, osteoarthritis, rheumatoid arthritis, metastatic bone disease, Gaucher's disease, avascular necrosis, polyostotic fibrous dysplasia, Charcot's joint, glucocortoid-induced osteoporosis, osteogenesis imperfecta, homocystinuria, lysinuric protein intolerance, Turner's syndrome, immobilization, fibrous dysplasia, fibrogenesis imperfecta ossium, periodontal disease, tooth loss, hypercalcemia of malignancy, and multiple myeloma.

The method of the present invention may be further characterized by orally administering to a mammal a composition containing a supplementary effective amount of a non-activated metabolite of vitamin D₂ and/or D₃, and a pharmaceutically effective amount of bisphosphonate, as a unit dosage, according to a continuous schedule having a dosing interval of once-weekly, twice-weekly, bi-weekly, once-monthly, and once bi-monthly. The method is suitable for preventing or treating metabolic bone disease, wherein the continuous schedule is maintained until the desired therapeutic effect is achieved.

The term “vitamin D,” as used herein, means non-activated forms of both vitamin D₂ (ergocalciferol) and vitamin D₃ (cholecalciferol).

The phrase “a non-activated metabolite of vitamin D₂ and/or D₃,” as used herein, means hydroxylated forms of vitamin D₂ (ergocalciferol) and vitamin D₃ (cholecalciferol), e.g. 25-hydroxy-cholecalciferol, and 24,25-dihydroxy-cholecalciferol. These non-activated metabolites are the primary storage form of vitamin D₃ in the human body. 25-Hydroxy-cholecaliferol may be further hydroxylated in the body to form 1,25-dihydroxy-cholecalciferol (an active precursor of vitamin D₃).

The term “IU,” as used herein, means International Units. One microgram of vitamin D is approximately 40 International Units.

The term “supplementary effective amount,” as used herein, means an exogenous amount of vitamin D₂ and/or D₃ metabolites, wherein the amount is sufficient for reducing vitamin D deficiency in mammals, and particularly mammals suffering from abnormal bone resorption.

The term “pharmaceutically effective amount,” as used herein, means that a pharmaceutical composition suitable for inhibiting bone resorption in mammals, where the composition is characterized as containing a supplementary effective amount of a non-activated metabolite of vitamin D₂ and/or D₃, and a pharmaceutically effective amount of at least one bisphosphonate as a unit dosage according to a continuous schedule having a dosing interval of once-weekly dosing.

The term “abnormal bone resorption,” as used herein means a degree of bone resorption that exceeds the degree of bone formation, either locally, or in the skeleton as a whole. Alternatively, “abnormal bone resorption” can be associated with the formation of bone having an abnormal structure.

The term “bone resorption inhibiting,” as used herein, means treating or preventing bone resorption by the direct or indirect alteration of osteoclast formation or activity. Inhibition of bone resorption refers to treatment or prevention of bone loss, especially the inhibition of removal of existing bone either from the mineral phase and/or the organic matrix phase, through direct or indirect alteration of osteoclast formation or activity.

The terms “a mammal in need of treatment,” “a mammal in need of prevention,” “a mammal in need thereof,” and “a mammal at risk thereof” refer to a mammal in need of treatment for a disease condition, in need of prevention of a disease condition, or at risk of developing a disease condition as determined by a clinician or researcher. The terms “a human in need of treatment,” “a human in need of prevention,” “a human in need thereof,” and “a human at risk thereof” refer to a human in need of treatment for a disease condition, in need of prevention of a disease condition, or at risk of developing a disease condition as determined by a clinician or researcher.

The terms “continuous schedule” or “continuous dosing schedule,” as used herein, mean that the dosing regimen is repeated until the desired therapeutic effect is achieved. The continuous schedule or continuous dosing schedule is distinguished from cyclical or intermittent administration.

The term “until the desired therapeutic effect is achieved,” as used herein, means that the bisphosphonate compound is continuously administered, according to the dosing schedule chosen, up to the time that the clinical or medical effect sought for the disease or condition is observed by the clinician or researcher.

The term “simultaneous administration,” as used herein, means administration of the components of the invention by combining the metabolite and the bisphosphonate components of the invention and administering the same as a combination composition.

The term “alternate administration,” as used herein, means separate administration of specific dosages of the components of the invention by separately administering dosages of the metabolites and bisphosphonate at different time intervals in accordance with a continuous administration schedule.

For methods of treatment of the present invention, the bisphosphonate compound is continuously administered until the desired change in bone mass or structure is observed. However, the method of treatment may be continued after the desired change in bone mass or structure is observed to maintain the effect thereof. In such instances, achieving an increase in bone mass or a replacement of abnormal bone structure with more normal bone structure are the desired objectives. For methods of prevention of the present invention, the bisphosphonate compound is continuously administered for as long as necessary to prevent the undesired condition. In such instances, maintenance of bone mass density is often the objective. Non-limiting examples of administration periods can range from about 2 weeks to the remaining life span of the mammal. For humans, administration periods can range from about 2 weeks to the remaining life span of the human; typically from about 2 weeks to about 30 years; preferably from about 1 month to about 20 years; more preferably from about 6 months to about 15 years; and most preferably from about 1 year to about 10 years.

Methods of the Present Invention

The present invention may be characterized as methods for inhibiting bone resorption and treating abnormal bone resorption in mammals.

The present invention may be further characterized as a continuous dosing schedule for the treatment or prevention of metabolic bond disease, whereby a pharmaceutical composition characterized as a unit dosage containing a supplementary effective amount of non-activated vitamin D₂ and/or D₃ metabolites and a pharmaceutically effective amount of at least one bisphosphonate. The composition may be regularly administered according to a dosing interval of once-weekly, twice-weekly, biweekly, once-monthly, and bimonthly.

The invention may also be characterized as a method of preventing or treating metabolic bone disease, characterized by concomitantly administering to a mammal, in need thereof, a single unit dosage of a supplementary effective amount of a non-activated metabolite of vitamin D₂ and/or D₃, and a single unit dosage of a pharmaceutically effective amount of at least one bisphosphonate, wherein administration is simultaneously or alternately, on a continuous dosing schedule. The administration may be according to dosing intervals of once-weekly, twice-weekly, bi-weekly, once-monthly, and bimonthly. The combined action of concomitantly administering the non-active metabolites and bisphosphonate, either simultaneously or alternately, on a continuous schedule is advantageous in preventing or treating metabolic bond disease, and it provides the synergistic effect of administering both the metabolite and bisphosphonate in a single unit dosage form.

For example, when referring to ‘once-weekly dosing’, it is meant that a unit dosage of bisphosphonate and metabolites of vitamin D₂ and/or D₃ is administered once a week, i.e. one time during a seven-day period, preferably on the same day of each week. In the once-weekly dosing regimen, the unit dosage is generally administered about every seven days. A non-limiting example of a once-weekly dosing regimen would entail the administration of a unit-dosage composition of the bisphosphonate and a non-activated metabolite of vitamin D₂ and/or D₃ every Sunday. It is preferred that the unit dosage is not administered on consecutive days, but the once-weekly dosing regimen can include a dosing regimen in which unit dosages are administered on two consecutive days falling within two different weekly periods.

The methods and compositions of the present invention are useful for inhibiting bone resorption and for treating or preventing abnormal bone resorption and conditions associated therewith. Such conditions include both generalized and localized bone losses. Also, the creation of bone having an abnormal structure, as in Paget's disease, can be associated with abnormal bone resorption.

The methods and compositions of the present invention are useful for treating or preventing conditions such as metastatic bone disease, hypercalcemia of malignancy, and multiple myeloma. Metastatic bone disease involves tumor-induced skeletal metastases which commonly result from breast cancer, prostate cancer, lung cancer, renal cancer, thyroid cancer, and multiple myeloma. The most frequent clinical manifestations of bone metastases are pain, pathological fracture, immobility, nerve root or spinal cord compression, hypercalcemia, and compromised hematopoiesis. Hypercalcemia of malignancy is also tumor-induced. It is characterized by high levels of serum calcium and is often associated with metastatic bone disease, particularly with non-ambulatory subjects. Multiple myeloma is a primary tumor of the bone marrow cells. See U.S. Pat. No. 5,780,455 to Brenner et al., issued Jul. 14, 1998, which is incorporated by reference herein in its entirety.

The term “generalized bone loss” means bone loss at multiple skeletal sites or throughout the skeletal system. The term “localized bone loss” means bone loss at one or more specific, defined skeletal sites.

Generalized boss loss leads to osteopenia and osteoporosis. Osteoporosis is most common in post-menopausal women, wherein estrogen production has been greatly diminished. However, osteoporosis can also be steroid-induced and has been observed in males due to age, hypogonadism, and other causes. Osteoporosis can be induced by disease, e.g. rheumatoid arthritis, it can be induced by secondary causes, e.g. glucocorticoid therapy, or it can come about due to an identifiable cause, i.e. idiopathic osteoporosis. In the present invention, preferred methods include the treatment or prevention of abnormal bone resorption in osteoporotic humans.

Localized bone loss has been associated with periodontal disease, with bone fractures, and with periprosthetic osteolysis (in other words where bone resorption has occurred in proximity to a prosthetic implant).

Generalized or localized bone loss can occur from disuse, which is often a problem for those confined to a bed or a wheelchair, or for those who have an immobilized limb set in a cast or in traction.

It has also been shown that the incidence of vertebral fractures can be reduced when an effective amount of alendronate is administered over a substantial period of time. The decrease in the risk of vertebral fractures is estimated to be at least about 40%, preferably at least about 45%, and even more preferably at least about 48%; this decrease was found to be statistically significant (when compared to placebo). When the total number of vertebral fractures (as opposed to the number of patients with fractures) was calculated, alendronate produces at least about 50%, preferably at least about 60% and even more preferably at least about 63%.reduction in vertebral fracture rate per 100 patients when compared to placebo. Likewise, alendronate produces a statistically significant decrease in the progression of vertebral deformity as compared to placebo patients.

It has also been found that the increase in bone mineral density observed with the administration of alendronate is positively associated with a decrease in vertebral fractures, a decrease in spinal deformity and a retention of height. This indicates that when administered for a substantial period of time, alendronate not only decreases bone resorption, but also acts positively to produce a strengthened bone.

The patient that receives alendronate and a non-activated metabolite of vitamin D₂ and/or D₃ according to this invention is losing bone mass and may be expected to develop osteoporosis if left untreated.

The methods and compositions of the present invention are useful for treating and or preventing the following conditions or disease states: osteoporosis, including but not limited to, post-menopausal osteoporosis, steroid-induced osteoporosis, male osteoporosis, other disease-induced osteoporosis, idiopathic osteoporosis, and glucocorticoid-induced osteoporosis; Paget's disease; osteoarthritis, abnormally increased bone turnover; localized bone loss associated with periprosthetic bone loss or osteolysis; bone fractures; metastatic bone disease; Gaucher's disease, avascular necrosis, polyostotic fibrous dysplasia, Charcot's joint, parasitic disorders, osteogenesis imperfecta, homocystinuria, lysinuric protein intolerance, Turner's syndrome, immobilization, fibrous dysplasia, fibrogenesis imperfecta ossium, periodontal disease, tooth loss, hypercalcemia of malignancy; multiple myeloma; and osteopenia, including but not limited to, immobilization-induced osteopenia and osteopenia due to bone metastases.

The invention further relates to methods for inhibiting bone resorption, and treating abnormal bone resorption, characterized by orally administering to a mammal a pharmaceutical composition characterized as containing a supplementary effective amount of a non-activated metabolite of vitamin D₂ and/or D₃, wherein the supplementary effective amount may be from about 100 IU to about 60,000 IU, and a pharmaceutically effective amount of at least one bisphosphonate, pharmaceutically acceptable salts, derivatives, hydrates, and mixtures thereof, wherein the pharmaceutically effective amount may be from about 0.05 mg to about 560 mg, on an alendronic acid active weight basis, pharmaceutically acceptable salts, derivatives, hydrates, and mixtures thereof.

In another embodiment, the invention relates to methods for preventing Paget's disease, and for treating or preventing a disease selected from osteoporosis, and metastatic bone disease characterized by orally administering to a mammal a pharmaceutical composition containing a supplemental amount of a non-activated metabolite of vitamin D₂ and/or D₃, wherein the supplementary effective amount may be from about 100 IU to about 60,000 IU, and a pharmaceutically effective amount of at least one bisphosphonate, wherein the pharmaceutically effective amount of may be from about 0.05 mg to about 560 mg of bisphosphonate, pharmaceutically acceptable salts, derivatives, hydrates, and mixtures thereof, on an alendronic acid active weight basis, pharmaceutically acceptable salts, derivatives, hydrates, and mixtures thereof.

Bisphosphonates

The bisphosphonate of the present invention corresponds to the general chemical formula:

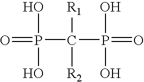

wherein R₁ is independently selected from H, OH and CI, and R₂ is independently selected CH₃, CI, CH₂CH₂NH₂, (CH₂)₃NH₂, CH₂-3-pyridine, CH₂-S-phenyl-Cl, CH₂CH₂N(CH₃)(pentyl), CH₂-imidazole, CH₂-2-imidazo-pyridinyl, N-(cycloheptyl), CH₂CH₂N(CH₃)₂, CH₂)₅NH₂, and CH₂-1-pyrrolidinyl, and combinations thereof.

Nonlimiting examples of bisphosphonates useful herein include the following:

Alendronic acid, 4-amino-1-hydroxybutylidene-1,1-bisphosphonic acid;

4-Amino-1-hydroxybutylidene-1,1-bisphosphonic acid monosodium trihydrate (alendronate);

Alendronate as described in U.S. Pat. No. 4,922,007, to Kieczykowski et al., issued May 1, 1990, and U.S. Pat. No. 5,019,651, to Kieczykowski, issued May 28, 1991, both of which are incorporated by reference herein in their entirety;

Cycloheptylaminomethylene-1,1-bisphosphonic acid, YM 175, Yamanouchi (incadronate or cimadronate), as described in U.S. Pat. No. 4,970,335, to Isomura et al., issued Nov. 13, 1990, which is incorporated by reference herein in its entirety;

1,1-Dichloromethylene-1,1-diphosphonic acid (clodronic acid), and the disodium salt (clodronate, Procter and Gamble), are described in Belgium Patent 672,205 (1966) and J. Org. Chem 32, 4111 (1967), both of which are incorporated by reference herein in their entirety;

1-Hydroxy-3-(1-pyrrolidinyl)-propylidene-1,1-bisphosphonic acid (EB-1053);

1-Hydroxyethane-1,1-diphosphonic acid (etidronic acid);

1-Hydroxy-3-(N-methyl-N-pentylamino)propylidene-1,1-bisphosphonic acid, also known as BM-210955, Boehringer-Mannheim (ibandronate), is described in U.S. Pat. No. 4,927,814, issued May 22, 1990, which is incorporated by reference herein in its entirety;

[1-Hydroxy-2-imidazopyridin-(1,2-a)-3-ylethylidene]-bisphosphonate (minodronate);

6-Amino-1-hydroxyhexylidene-1,1-bisphosphonic acid (neridronate);

3-(Dimethylamino)-1-hydroxypropylidene-1,1-bisphosphonic acid (olpadronate);

3-Amino-1-hydroxypropylidene-1,1-bisphosphonic acid (pamidronate);

[2-(2-Pyridinyl)ethylidene]-1,1-bisphosphonic acid (piridronate) is described in U.S. Pat. No. 4,761,406, which is incorporated by reference in its entirety;

1-Hydroxy-2-(3-pyridinyl)-ethylidene-1,1-bisphosphonic acid (risedronate);

(4-Chlorophenyl)thiomethane-1,1-bisphosphonic acid (tiludronate) as described in U.S. Pat. No. 4,876,248, to Breliere et al., Oct. 24, 1989, which is incorporated by reference herein in its entirety; and

1-Hydroxy-2-(1H-imidazol-1-yl)ethylidene-1,1-bisphosphonic acid (zoledronate).

In another embodiment of the invention, the bisphosphonate selected from alendronate, pharmaceutically acceptable salts, derivatives, hydrates thereof, and mixtures of salts, derivatives and hydrates.

In a class of the invention, the pharmaceutically acceptable salt of alendronate may be selected from sodium, potassium, calcium, magnesium, ammonium salts of alendronate, and mixtures thereof.

In a subclass of this class of the invention, the pharmaceutically acceptable salt of alendronate may be alendronate monosodium or a hydrate thereof.

In a subclass of this class of the invention, the pharmaceutically acceptable salt of alendronate may be selected from sodium, potassium, ammonium salts of alendronate, and mixtures thereof.

In another subclass of this class of the present invention, the pharmaceutically acceptable hydrate of alendronate monosodium may be selected from the monohydrate and the trihydrate.

In yet another subclass of this class of the present invention, the pharmaceutically acceptable hydrate of alendronate monosodium is the trihydrate.

In yet another subclass of this class of the present invention, the pharmaceutically acceptable hydrate of alendronate monosodium is the monohydrate.

As used throughout this specification and claims, the terms “alendronic acid” and “bisphosphonic acid” include the related bisphosphonic acid forms, pharmaceutically acceptable salt forms and equilibrium mixtures thereof.

The terms include crystalline, hydrated-crystalline, and amorphous forms of alendronic acid and pharmaceutically acceptable salts thereof. The term “alendronic acid” specifically includes, but is not limited to, anhydrous alendronate monosodium, alendronate monosodium hemihydrate, alendronate monosodium monohydrate, alendronate monosodium trihydrate, anhydrous alendronate dipotassium, and alendronate dipotassium pentahydrate. Alendronate monosodium monohydrate and other crystalline forms of alendronate sodium are disclosed in U.S. Pat. No. 6,281,381 to Finkelstein et al. issued Aug. 28, 2001, which is incorporated by reference herein in its entirety. Potassium salts of alendronic acid, and hydrates thereof, are disclosed in WO 99/20635, issued Apr. 29, 1999, which is incorporated by reference herein in its entirety.

Pharmaceutically acceptable salts, derivatives, and hydrates of the bisphosphonates are also useful herein. Nonlimiting examples of salts include those selected from alkali metal, alkaline metal, ammonium, and mono-, di-, tri-, or tetra-C₁-C₃₀-alkyl-substituted ammonium. Preferred salts are those selected from sodium, potassium, calcium, magnesium, and ammonium salts. Nonlimiting examples of derivatives include those selected from esters and amides. Also encompassed within the scope of the invention are the various hydrates and other solvates of the bisphosphonates, or pharmaceutically acceptable salts thereof. Also encompassed within the scope of the present invention are hydrates of alendronate, including but not limited to, hydrates with water content between about one to twelve percent, and their crystalline forms. Nonlimiting examples of hydrates include the dihydrate, hemihydrate, ¼ hydrate, ⅓ hydrate, ⅔ hydrate, ¾ hydrate, 5/4 hydrate, 4/3 hydrate, 3/2 hydrate, 5/3 hydrate, and 7/4 hydrates, which are described in U.S. Pat. No. 6,281,381, incorporated herein by reference thereto.

It should be noted that the terms “bisphosphonate” and “bisphosphonates”, as used herein in referring to the therapeutic agents are meant to also encompass diphosphonates, biphosphonic acids, and diphosphonic acids, as well as salts, derivatives and hydrates of these materials. The use of a specific nomenclature in referring to the bisphosphonate or bisphosphonates is not meant to limit the scope of the present invention, unless specifically indicated. Because of the nixed nomenclature currently in use by those or ordinary skill in the art, reference to a specific weight or percentage of a bisphosphonate compound in the present invention is on an acid active weight basis, unless indicated otherwise herein. For example, the phrase “about 70 mg of a bone resorption inhibiting bisphosphonate selected from alendronate, pharmaceutically acceptable salts, derivatives and hydrates thereof, and mixtures thereof, on an alendronic acid active weight basis” means that the amount of the bisphosphonate compound selected is calculated based on 70 mg of alendronic acid.

Vitamin D

The methods and compositions of the present invention may contain Vitamin D of the formulae:

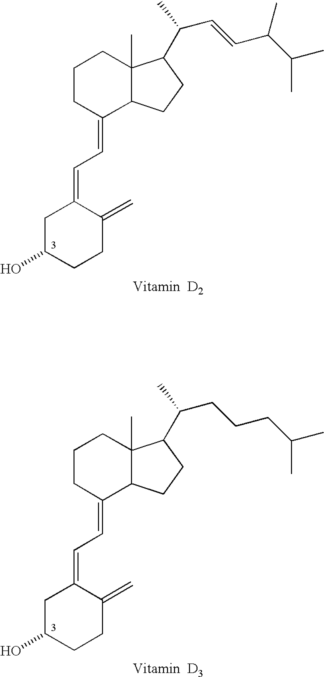

Vitamin D₂ and vitamin D₃ metabolites may be characterized as “storage” forms in mammals as 25-hydroxyvitamin D₂ (25-hydroxy-ergocalciferol) and 25-hydroxyvitamin D₃ (25-hydroxy-cholecalciferol), respectively.

As used herein 1 USP (or 1 IU) of vitamin D₃ is defined as the activity of 0.025 micrograms of vitamin D₃. For instance, 2800 IU of vitamin D₃ is equivalent to about 70 micrograms of vitamin D₃. Since 25-hydroxyvitamin D₃ is about 1.4 times (1.4×) more potent that 25-hydroxyvitamin D₃, 50 micrograms of 25-hydroxyvitamin D₃ would be equivalent to about 70 micrograms (2800 IU) of vitamin D₃.

Pharmaceutical Compositions

Pharmaceutical compositions useful in the present invention may be characterized as containing, in combination, a supplementary effective amount of a non-activated metabolite of vitamin D₂ and/or D₃, and a pharmaceutically effective amount of at least one bisphosphonate. When concomitantly administered, the composition may be characterized as separate compositions administered simultaneously or alternately. The bisphosphonate and vitamin D combination is typically administered in admixture with suitable pharmaceutical diluents, excipients, or carriers, collectively referred to herein as “carrier materials”, suitably selected with respect to oral administration, i.e. compressed, coated, or uncoated tablets, capsules, hard or gelatin capsules, pellets, elixirs, syrups, slurries, emulsions, suspensions, solutions, effervescent and effervescent-buffered compositions, powders, films, and the like, and consistent with conventional pharmaceutical practices. Effervescent compositions containing a bisphosphonate are disclosed in U.S. Pat. No. 5,853,759 to Katdare et al. and UK Patent No. 2153225 to Gentili Spa, both of which are incorporated herein by reference thereto. For example, for oral administration in the form of a tablet, capsule, pellet, or powder, the active ingredient can be combined with an oral, non-toxic, pharmaceutically acceptable inert carrier such as lactose, starch, sucrose, glucose, methyl cellulose, magnesium stearate, mannitol, sorbitol, croscarmellose sodium and the like; for oral administration in liquid form, e.g. elixirs, syrups, slurries, emulsions, suspensions, solutions, effervescent compositions, and effervescent-buffered compositions, the oral drug components can be combined with any oral, non-toxic, pharmaceutically acceptable inert carrier such as ethanol, glycerol, water and the like. Moreover, when desired or necessary, suitable binders, lubricants, disintegrating agents, buffers, coatings, and coloring agents can also be incorporated. Suitable binders can include starch, gelatin, natural sugars such a glucose, anhydrous lactose, free-flow lactose, beta-lactose, and corn sweeteners, natural and synthetic gums, such as acacia, guar, tragacanth or sodium alginate, carboxymethyl cellulose, polyethylene glycol, waxes, and the like. Lubricants used in these dosage forms include sodium oleate, sodium stearate, magnesium stearate, sodium benzoate, sodium acetate, sodium chloride and the like.

A particularly preferred tablet formulation for alendronate monosodium trihydrate is that described in U.S. Pat. No. 5,358,941, to Bechard et al., issued Oct. 25, 1994, which is incorporated by reference herein in its entirety. The compounds used in the present method can also be coupled with soluble polymers as targetable drug carriers. Such polymers can include polyvinylpyrrolidone, pyran copolymer, polyhydroxylpropyl-methacrylamide, and the like.

The precise dosage of the bisphosphonate and a non-activated metabolite of vitamin D₂ and/or D₃ formulation will vary with the dosing schedule or interval. The oral potency of the particular bisphosphonate chosen will depend upon the age, size, sex and condition of the mammal or human, the nature and severity of the disorder to be treated, and other relevant medical and physical factors. Thus, a precise pharmaceutically effective amount cannot be specified in advance but can be readily determined by the caregiver or clinician. Appropriate amounts can be determined by routine experimentation from animal models and human clinical studies. Generally, an appropriate amount of bisphosphonate is chosen to obtain a bone resorption inhibiting effect, i.e. a bone resorption inhibiting amount of the bisphosphonate is administered. For humans, an effective oral dose of bisphosphonate typically ranges from about 0.0001 mg/kg to about 100 mg/kg body weight; and preferably from about 0.0005 to about 20 mg/kg of body weight for a 75 kg subject.

Generally, an appropriate supplementary amount of a non-activated metabolite of vitamin D₂ and/or D₃ may be chosen to supplement vitamin D nutrition during the dosing interval. For humans, the supplementary amount of non-activated metabolite of vitamin D₂ and/or D₃ may range from about 100 to about 60,000 μl/day; typically, the supplementary amount of a non-activated metabolite of vitamin D₂ and/or D₃ may range from about 200 to about 40,000 IU/day; and preferably, the supplemental amount of a non-activated metabolite of vitamin D₂ and/or D₃ may range from about 400 to about 1,200 IU/day. However, the maximum amount of vitamin D generally should not exceed about 100,000 IU/administration. As high as 4,000 IU/day, as well as, doses higher than 4,000 IU may be given at less frequent intervals due to the long body half-life of vitamin D (See Vieth R. et al., Efficacy and safety of vitamin D₃ intake exceeding the lowest observed adverse effect level, Am J Clin Nutr (2001); 73; 288-94; and Vieth R. Vitamin D Supplementation, 25-hydroxy vitamin D concentration, and safety, Am J Clin Nutr 1999; 69: 842-856). Unless otherwise noted, all vitamin D dosages are based on the interval, i.e. days, between dosing. For example, if 200 IU/day of vitamin D is desired and the dosing period is weekly (based on 7 days/week), the weekly dosing would be 200 Iu/day×7 days/week or 1,400 IU/week of vitamin D.

For humans, compositions characterized as containing alendronate, pharmaceutically acceptable salts, derivatives and hydrates thereof, and a non-activated metabolite of vitamin D₂ and/or D₃, a general oral supplemental amount of a non-activated metabolite of vitamin D₂ and/or D₃ containing from about 100 IU to about 60,000 IU are contemplated. Non-limiting examples of an oral supplementary amount of a non-activated metabolite of vitamin D₂ and/or D₃ include, but are not limited to, dosages of 1,400 IU, 2,800 IU, 4,200 IU, 5600 IU, 7,000 IU, 8,400 IU, 14,000 IU, and 28,000 IU in combination with varying amounts of bisphosphonate. For humans, compositions characterized as containing a non-activated metabolite of vitamin D₂ and/or D₃ and a pharmaceutically effective amount of alendronate, pharmaceutically acceptable salts, derivatives and hydrates thereof, typically containing from about 0.05 to about 560 mg, on an alendronic acid active weight basis are contemplated. Non-limiting examples of a pharmaceutically effective amount of alendronate include, but are not limited to, dosages of about 2.5 mg, 5 mg, 8.75 mg, 10 mg, 17.5 mg, 35 mg, 40 mg, 70 mg, 140 mg, 280 mg, and 560 mg of alendronate, on an alendronic acid active weight basis, pharmaceutically acceptable salts, derivatives, hydrates, and mixtures thereof, in combination with varying amounts of a non-activated metabolite of vitamin D₂ and/or D₃.

For once-weekly dosing, a pharmaceutically effective amount of alendronate comprises from about 17.5 mg to about 280 mg of the alendronate, on an alendronic acid active weight basis, pharmaceutically acceptable salts, derivatives, hydrates, and mixtures thereof. Examples of weekly pharmaceutically effective amount of alendronate for preventing osteoporosis include, but are not limited to, unit dosages of from about 35 to about 70 mg of the alendronate, on an alendronic acid active weight basis; a unit dosage useful for treating osteoporosis may be at least about 70 mg of the alendronate; a unit dosage which is useful for treating Paget's disease may be at least about 280 mg of the alendronate, on an alendronic acid active weight basis, pharmaceutically acceptable salts, derivatives, hydrates, and mixtures thereof; and a unit dosage useful for treating metastatic bone disease may be at least about 280 mg of the alendronate, on an alendronic acid active weight basis, pharmaceutically acceptable salts, derivatives, hydrates, and mixtures thereof.

In another embodiment of the present invention, the composition comprises a supplementary amount of a non-activated metabolite of vitamin D₂ and/or D₃, wherein the supplementary amount is from about 100 IU to about 60,000 IU, and of a pharmaceutically effective amount of alendronate, pharmaceutically acceptable salts, hydrates, derivatives, and mixtures thereof, wherein the pharmaceutically effective amount of alendronate is from about 0.05 mg to about 280 mg, on an alendronic acid active weight basis.

Even though it is conventional to dose and calculate the dosages of bisphosphonates on the basis of bisphosphonic acid active weight basis, bisphosphonate dosages can be calculated and administered based on other salt or hydrate forms. For example, risedronate dosages are calculated based on the weight of the anhydrous risedronate sodium salt. Each tablet of risedronate contains the equivalent of 5 mg or 30 mg of anhydrous risedronate sodium, in the form of the hemi-pentahydrate with small amounts of monohydrate according to the Physician's Desk Reference, (55th Edition, page 2664, (2001)), which is incorporated herein in its entirety. Bisphosphonate doses calculated on the basis of their salt, derivative or hydrate forms are included within the dosage ranges of the present invention on the basis of their bisphosphonic acid active weights. Additionally, the doses of all hydrate forms of alendronate are calculated on the basis of the alendronic acid active weight. For instance, the doses of the monohydrate, trihydrate, hemihydrate and all other hydrate forms of alendronate and its salts, are calculated on the basis of their alendronic acid active weights.

Nonlimiting examples of oral compositions comprising alendronate, as well as other bisphosphonates, and a non-activated metabolite of vitamin D₂ and/or D₃, are illustrated in the examples below.

The present invention also provides for a composition characterized as containing a supplementary amount of a non-activated metabolite of vitamin D₂ and/or D₃, and a pharmaceutically effective amount of at least one bisphosphonate, or pharmaceutically acceptable salt, derivative or hydrate thereof, and one or more active ingredients for the manufacture of a medicament for the treatment or prevention of the following conditions or disease states: osteoporosis treatment or prevention, including but not limited to, post-menopausal osteoporosis, steroid-induced osteoporosis, male osteoporosis, disease-induced osteoporosis, idiopathic osteoporosis, and glucocorticoid-induced osteoporosis; osteoarthritis; rheumatoid arthritis; Paget's disease; osteoarthritis; abnormally increased bone turnover; localized bone loss associated with periprosthetic bone loss or osteolysis; bone fractures; metastatic bone disease; Gaucher's disease, avascular necrosis, polyostotic fibrous dysplasia, Charcot's joint, parasitic disorders, osteogenesis imperfecta, homocystinuria, lysinuric protein intolerance, Turner's syndrome, immobilization, fibrous dysplasia ossificans progressiva, fibrogenesis imperfecta ossium, periodontal disease, tooth loss, hypercalcemia of malignancy; multiple myeloma; and osteopenia, including but not limited to, immobilization-induced osteopenia and osteopenia due to bone metastases.

In further embodiments, the methods and compositions of the present invention can also comprise a histamine H2 receptor blocker (i.e. antagonist) and/or a proton pump inhibitor. Histamine H2 receptor blockers and proton pump inhibitors are well known therapeutic agents for increasing gastric pH. See L.J. Hixson, et al., Current Trends in the Pharmacotherapy for Peptic Ulcer Disease, Arch. Intern. Med., vol. 152, pp. 726-732 (April 1992), which is incorporated by reference herein in its entirety. It is found in the present invention that the sequential oral administration of a histamine H2 receptor blocker and/or a proton pump inhibitor, followed by a bisphosphonate and a non-activated metabolite of vitamin D₂ and/or D₃ composition can help to minimize adverse gastro-intestinal effects. In one embodiment of the present invention, the histamine H2 receptor blocker and/or proton pump inhibitor is administered from about 30 minutes to about 24 hours prior to the administration of the bisphosphonate and a non-activated metabolite of vitamin D₂ and/or D₃ composition. In a class of this embodiment, the histamine H2 receptor blocker and/or proton pump inhibitor is administered from about 30 minutes to about 12 hours prior to the administration of the bisphosphonate and a non-activated metabolite of vitamin D₂ and/or D₃ composition.

The dosage of the histamine H2 receptor blocker and/or proton pump inhibitor depends upon the particular compound selected and factors associated with the mammal to be treated, i.e. size, health, etc.

Nonlimiting examples of histamine H2 receptor blockers and/or proton pump inhibitors include those selected from cimetidine, famotidine, nizatidine, ranitidine, omprazole, and lansoprazole.

One embodiment of the invention relates to a method for preventing or treating abnormal bone resorption in a mammal, in need thereof, characterized by orally administering to the mammal a pharmaceutical composition, as a unit dosage, containing a supplementary effective amount of from about 100 to about 60,000 IU of a non-activated metabolite of vitamin D₂ and/or D₃, and a pharmaceutically effective amount of from about 0.05 to about 280 mg of alendronate, on an alendronic acid active weight basis, pharmaceutically acceptable salts, derivatives, hydrates, and mixtures thereof, wherein the dosing interval is once-weekly.

In a more specific embodiment, the invention relates to a pharmaceutical composition suitable for oral administration for the treatment or prevention of abnormal bone resorption in mammals, characterized as a unit dosage of a supplementary effective amount of at least about 2,800 IU of a non-activated metabolite of vitamin D₂ and/or D₃, and a pharmaceutically effective amount of at least about 70 mg of alendronate, on an alendronic acid active weight basis, wherein the dosing interval is once-weekly. The invention also relates to a method of preventing or treating abnormal bone resorption in a mammal, in need thereof, comprising orally administering to said mammal a pharmaceutical composition, characterized as a unit dosage of a supplementary effective amount of at least about 2,800 IU of a non-activated metabolite of vitamin D₂ and/or D₃ and a pharmaceutically effective amount of at least about 70 mg of alendronate, on an alendronic acid weight active weight basis, wherein the dosing interval is once-weekly.

In another embodiment, the invention relates to a pharmaceutical composition suitable for oral administration for the treatment or prevention of abnormal bone resorption in mammals, in need thereof, characterized as a unit dosage of a supplementary effective amount of at least about 5,600 IU of a non-activated metabolite of vitamin D₂ and/or D₃, and a pharmaceutically effective amount of at least about 70 mg of alendronate, on an alendronic acid active weight basis, wherein the dosing interval is once-weekly. The invention also relates to a method of preventing or treating abnormal bone resorption in a mammal, in need thereof, characterized as orally administering to said mammal a pharmaceutical composition, containing a unit dosage of a supplementary effective amount of at least about 5,600 IU of a non-activated metabolite of vitamin D₂ and/or D₃ and a pharmaceutically effective amount of at least about 70 mg of alendronate, on an alendronic acid active weight basis, wherein the dosing interval is once-weekly.

In still another embodiment, the invention relates to a pharmaceutical composition suitable for oral administration for the treatment or prevention of abnormal bone resorption in mammals, in need thereof, characterized as containing a dosage of a supplementary effective amount of at least about 2,800 IU of a non-activated metabolite of vitamin D₂ and/or D₃, and a pharmaceutically effective amount of at least about 35 mg of alendronate, on an alendronic acid active weight basis, wherein the dosing interval is once-weekly. The invention also relates to a method of preventing or treating abnormal bone resorption in a mammal, in need thereof, characterized as orally administering to said mammal a pharmaceutical composition, containing a unit dosage of a supplementary effective amount of at least about 2,800 IU of a non-activated metabolite of vitamin D₂ and/or D₃ and a pharmaceutically effective amount of at least about 35 mg of alendronate, on an alendronic acid active weight basis, wherein the dosing interval is once-weekly.

In still yet another embodiment, the invention relates to a pharmaceutical composition suitable for oral administration for the treatment or prevention of abnormal bone resorption in mammals, in need thereof, containing a unit dosage of a supplementary effective amount of at least about 5,600 IU of a non-activated metabolite of vitamin D₂ and/or D₃, and a pharmaceutically effective amount of at least about 35 mg of alendronate, on an alendronic acid active weight basis, wherein the dosing interval is once-weekly. The invention also relates to a method of preventing or treating abnormal bone resorption in a mammal, in need thereof, characterized as orally administering to said mammal a pharmaceutical composition, containing a unit dosage of a supplementary effective amount of at least about 5,600 IU of a non-activated metabolite of vitamin D₂ and/or D₃ and a pharmaceutically effective amount of at least about 35 mg of alendronate, on an alendronic acid active weight basis, wherein the dosing interval is once-weekly.

Treatment Kits

In further embodiments, the present invention relates to a kit for conveniently and effectively carrying out the methods in accordance with the present invention. The kit may provide the non-activated metabolites of vitamin D₂ and/or D₃, and the bisphosphonate as a combination pharmaceutical composition for unit dosing, or for concomitant administration, simultaneously or alternately, wherein the components are separately packaged.

Such kits are especially suited for the delivery of solid oral forms, e.g. tablets or capsules, and liquid oral forms, e.g. liquids and emulsions. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered. Alternatively, placebo dosages, or calcium or dietary supplements, either in a form similar to or distinct from the bisphosphonate and non-activated metabolites of vitamin D₂ and/or D₃ unit dosages, can be included to provide a kit in which a dosage is taken every day. In those embodiments including a histamine H2 receptor and/or proton pump inhibitor, these agents can be included as part of the kit.

### EXAMPLES

The following examples further describe and demonstrate embodiments within the scope of the present invention. The examples are given solely for the purpose of illustration and are not to be construed as limitations of the present invention as many variations thereof are possible without departing from the spirit and scope of the invention.

### Example 1

### Effect of Vitamin D₃ on Alendronate Absorption

To examine the potential for an interaction between alendronate and vitamin D₃ metabolite administered orally, fourteen healthy adult subjects (6 men, 8 women, ages 33-61 yr.) were administered single 70-mg tablets of alendronate alone and together with a powdered dose of vitamin D₃ metabolite (5600 IU) suspended in 240 mL of water. This study was of an open, randomized, crossover two-way design. The purpose of the study was to obtain a preliminary estimate of the relative bioavailability of alendronate following a 70-mg tablet administered with vitamin D₃ metabolite, relative to alendronate administered alone.

Alendronate was administered orally as the 70-mg tablet in each of the 2 periods. In 1 period, the tablet was administered with vitamin D₃ metabolite powder reconstituted in plain tap water and in the alternate period the tablet was taken alone with plain tap water. Urine was collected for two hours preceding and 36 hours following each dose of alendronate for analytical determination of excreted alendronate. Relative bioavailability was estimated based on total urinary recovery of alendronate over the 36 hours postdose.

Urinary recovery of alendronate following the dose of 70-mg alone was 202 μg with a 90% CI of (126 μg, 279 μg), recoveries following the 70-mg dose administered together with vitamin D₃, averaged 238 μg with a 90% CI of (159 μg, 316 μg). The geometric mean ratio (90% CI) was estimated at 1.18 (0.80, 1.74).

Based on this investigation, oral administration of vitamin D₃ together with an oral dose of alendronate has minimal to no effect on the bioavailability of alendronate.

### Example 2

### Once-Weekly Dosing Regimen

Treatment of Osteoporosis.

Alendronate and vitamin D₃ metabolites tablets, liquid and effervescent formulations, and effervescent-buffered formulations containing about 70 mg of alendronate, on an alendronic acid active weight basis, and about 5,600 IU of vitamin D₃ metabolites are prepared (see Examples 4 and 5). The tablets or liquid formulations are orally administered to a subject once-weekly, i.e. preferably about once every seven days (for example, every Sunday), for a period of at least one-year. This method of administration is useful and convenient for treating osteoporosis while providing vitamin D nutrition. This method is also useful for improving subject acceptance and compliance, and ensuring that all subjects taking a bisphosphonate receive supplemental vitamin D nutrition.

Prevention of Osteoporosis

Alendronate and vitamin D₃ metabolite tablets or liquid formulations containing about 35 mg to about 70 mg of alendronate, on an alendronic acid active weight basis, and 5,600 IU of vitamin D₃ metabolite are prepared (see Examples 4 and 5). The tablets or liquid formulations are orally administered to a human subject once-weekly, i.e. preferably about once every seven days (for example, every Sunday), for a period of at least one-year. This method of administration is useful and convenient for preventing osteoporosis while providing vitamin D nutrition. This method is also useful for improving subject acceptance and compliance, and ensuring that all subjects taking a bisphosphonate receive supplemental vitamin D nutrition.

Alendronate and vitamin D₃ metabolite tablets or liquid formulations containing about 35 mg of alendronate, on an alendronic acid active weight basis, and 5,600 IU of vitamin D₃ metabolite are prepared (see Examples 4 and 5). The tablets or liquid formulations are orally administered to a human subject once-weekly, i.e. preferably about once every seven days (for example, every Sunday), for a period of at least one-year. This method of administration is useful and convenient for preventing osteoporosis while providing vitamin D nutrition. This method is also useful for improving subject acceptance and compliance, and ensuring that all subjects taking a bisphosphonate receive supplemental vitamin D nutrition.

Treatment of Paget's Disease

Alendronate and vitamin D₃ tablets or liquid formulations containing about 280 mg of alendronate, on an alendronic acid active weight basis, and about 5,600 IU of vitamin D₃ metabolite are prepared (see Examples 4 and 5). The tablets or liquid formulations are orally administered to a subject once-weekly, i.e. preferably about once every seven days (for example, every Sunday), for a period of at least one to six months. This method of administration is useful and convenient for treating Paget's disease while providing vitamin D nutrition. This method is also useful for improving subject acceptance and compliance, and ensuring that all subjects taking a bisphosphonate receive supplemental vitamin D nutrition.

Treatment of Metastatic Bone Disease

Alendronate and vitamin D₃ metabolite tablets or liquid formulations containing about 280 mg of alendronate, on an alendronic acid active weight basis, and 5,600 IU of vitamin D₃ metabolite are prepared (see Examples 4 and 5). The tablets or liquid formulations are orally administered to a subject once-weekly, i.e. preferably about once every seven days (for example, every Sunday). This method of administration is useful and convenient for treating metastatic bone disease while providing vitamin D nutrition. This method is also useful for improving subject acceptance and compliance, and ensuring that all subjects taking a bisphosphonate receive supplemental vitamin D nutrition.

### Example 3

### Alendronate and Vitamin D₃ Metabolite Tablets or Liquid Formulations

In further embodiments, alendronate and vitamin D₃ metabolite tablets or liquid formulations are orally dosed, at the desired dosage, according to the dosing schedule of Example 2, for treating or preventing other disorders associated with abnormal bone resorption.

### Example 4

### Bisphosphonate and Vitamin D₃ Tablets

Bisphosphonate and vitamin D₃ metabolite containing tablets are prepared using standard mixing and formation techniques as described in U.S. Pat. No. 5,358,941, to Bechard et al., issued Oct. 25, 1994, which is incorporated by reference herein in its entirety.

Tablets containing about 35 mg of alendronate and 5,600 IU of vitamin D₃ metabolite, on an alendronic acid active basis, are prepared using the following relative weights of ingredients:

| Ingredient | Per Tablet | Per 4000 Tablets |
|---|---|---|
| Alendronate Monosodium | 45.68 mg | 182.72 g |
| Trihydrate | | |
| Vitamin D₃ metabolite | 140 μg | 560 mg |
| Anhydrous Lactose, NF | 71.32 mg | 285.28 g |
| Microcrystalline Cellulose, | 80.0 mg | 320.0 g |
| NF | | |
| Magnesium Stearate, NF | 1.0 mg | 4.0 g |
| Croscarmellose Sodium, NF | 2.0 mg | 8.0 g |

The resulting tablets are useful for administration in accordance with the methods of the present invention for inhibiting bone resorption, for preventing or treating bone resorption, for preventing or treating osteoporosis, and for treating Paget's disease.

Similarly, tablets comprising other relative weights of alendronate, on an alendronic acid active weight basis are prepared including, but not limited to, about 2.5 mg, 5 mg, 8.75 mg, 17.5 mg, 70 mg, 140 mg, 280 mg, and 560 mg per tablet. Similarly, tablets comprising other relative weights of vitamin D₃ metabolites per unit dosage are prepared including, but not limited to, about 1,400, 2,800, 5,600, 7,000 IU, 8,400 IU, 14,000 IU, or 28,000 IU per 75 mL volume.

### Example 5

### Liquid Bisphosphonate and Vitamin D₃ Metabolite Formulation

Liquid bisphosphonate and vitamin D₃ metabolite formulations are prepared using standard mixing techniques.

A liquid formulation containing about 70 mg of alendronate monosodium trihydrate, on an alendronic acid active weight basis, and 5600 IU of vitamin D₃ metabolite per about 75 mL of liquid is prepared using the following relative weights of ingredients:

| Ingredient | Weight |
|---|---|
| Alendronate Monosodium | 91.35 mg |
| Trihydrate | |
| Vitamin D₃ metabolite | 5600 IU (140 μg) |
| Sodium Citrate Dihydrate | 1500 mg |
| 1 N Sodium Hydroxide (aq) | qs pH 6.75 |
| Water | qs 75 mL |

Additional agents such as cosolvents, flavoring agents, coloring agents, preservatives, and stabilizers may also be specifically incorporated into the formulation as follows:

| Ingredient | Weight |
|---|---|
| Sodium Propylparaben | 22.5 mg |
| Sodium Butylparaben | 7.5 mg |
| Citric Acid Anhydrous | 56.25 mg |
| Sodium Saccharin | 7.5 mg |

The resulting liquid formulations are useful for administration in accordance with the methods of the present invention for inhibiting bone resorption, for preventing or treating bone resorption, for preventing or treating osteoporosis, and for treating Paget's disease.

Similarly, liquid formulations comprising other relative weights of alendronate, on an alendronic acid active weight basis, per unit dosage are prepared including, but not limited to, about 2.5 mg, 5 mg, 8.75 mg, 17.5 mg, 70 mg, 140 mg, and 280 mg per 75 mL volume. Similarly, liquid formulations comprising other relative weights of vitamin D₃ metabolites per unit dosage are prepared including, but not limited to, about 1,400, 2,800, 5,600, 7,000 IU, 8,400 IU, 14,000 IU, 28,000 or 5,600 IU per 75 mL volume. Also, the liquid formulations are prepared to provide other volumes for the unit dosage including, but not limited to, 135 ML, 240 mL and 480 mL volumes.

### Example 6

### Liquid Alendronate and Vitamin D₃ Metabolite Formulation

A liquid formulation containing 140 mg of alendronate monosodium trihydrate, on an alendronic acid active basis, in about 75 mL of liquid may be prepared using the following relative weights of ingredients:

| | |
|---|---|
| Alendronate monosodium trihydrate* | 2.437 mg/mL |
| Sodium Citrate | 21.18 mg/mL |
| Sodium hydroxide | qs pH 6.8 |
| Hydrochloric acid | qs pH 6.8 |
| Purified Water | qs 1.00 mL |

Additional agents such as cosolvents, flavoring agents, coloring agents, preservatives, and stabilizers may also be specifically incorporated in the formulation as follows:

| | |
|---|---|
| Alendronate monosodium trihydrate* | 2.437 mg/mL |
| Sodium Citrate | 21.18 mg/mL |
| Sodium propylparaben | 0.2250 mg/mL |
| Sodium butylparaben | 0.07500 mg/mL |
| Sodium saccharin | 0.100 mg/mL |
| Flavor | qs for taste |
| Sodium hydroxide | qs pH 6.8 |
| Hydrochloric acid | qs pH 6.8 |
| Purified Water | qs 1.00 mL |

*Corresponds to 1.867 mg free alendronic acid equivalents or 140 mg/75 mL

Method of Manufacture:

The specified amounts of the following ingredients may be added to purified water and mixed sequentially until dissolved: sodium propylparaben, sodium butylparaben, sodium citrate dihydrate, sodium saccharin, raspberry flavor, and alendronate monosodium trihydrate. The pH may be checked to target about pH 6.8 (range: about 6.4 to about 7.2). If required, the pH may be adjusted to 6.8 with aqueous sodium hydroxide or aqueous hydrochloric acid. The batch may be adjusted to final weight with purified water and filtered through a suitable filter (<50 μm). The solution may then be dispensed into suitable containers and capped. The aqueous solution can be used as is directly from the bottle or poured into a suitable container for dosing. After preparation of the liquid, 140 mg alendronate formulation, a non-activated metabolite of vitamin D₂ and/or D₃, specific supplementary amounts, e.g. but not limited to, about 1,400, 2,800, 5,600, 7,000 IU, 8,400 IU, 14,000 IU, 28,000 or 5,600 IU per 75 mL volume, may be combined with or concomitant administered with the alendronate.

### Example 7

### Twice-Weekly Dosing Regimen for Treatment of Paget's Disease of Bone

The alendronate-vitamin D formulation from Example 6 containing about 140 mg of alendronate in about 75 mL of liquid, on an alendronic acid active basis, may be orally administered to a human patient twice-weekly, preferably once every three or four days (for example, every Sunday and Wednesday), for a period of at least one to six months. This method of administration is useful and convenient for treating Paget's disease of bone, particularly in humans with difficulty in swallowing tablets, while minimizing adverse upper gastrointestinal effects, particularly esophageal irritation. This method is also useful for improving patient acceptance and compliance.

### Example 8

### Liquid Alendronate and Vitamin D₃ Metabolite Formulation

A liquid formulation containing 280 mg of alendronate monosodium trihydrate, on an alendronic acid active basis, in about 75 mL of liquid may be prepared using the following relative weights of ingredients:

| | |
|---|---|
| Alendronate monosodium trihydrate* | 4.873 mg/mL |
| Sodium Citrate | 21.18 mg/mL |
| Sodium hydroxide | qs pH 6.8 |
| Hydrochloric acid | qs pH 6.8 |
| Purified Water | qs 1.00 mL |

Additional agents such as cosolvents, flavoring agents, coloring agents, preservatives, and stabilizers may also be specifically incorporated in the formulation as follows:

| | |
|---|---|
| Alendronate monosodium trihydrate* | 4.873 mg/mL |
| Sodium Citrate | 21.18 mg/mL |
| Sodium propylparaben | 0.2250 mg/mL |
| Sodium butylparaben | 0.07500 mg/mL |
| Sodium saccharin | 0.100 mg/mL |
| Flavor | qs for taste |
| Sodium hydroxide | qs pH 6.8 |
| Hydrochloric acid | qs pH 6.8 |
| Purified Water | qs 1.00 mL |

*

The method of manufacture the alendronate formulation may be the same as that for Example 6. Thereafter, non-activated metabolites of vitamin D₂ and/or D₃, specific supplementary amounts, e.g. but not limited to, about 1,400, 2,800, 5,600, 7,000 IU, 8,400 IU, 14,000 IU, 28,000 or 5,600 IU per 75 mL volume, may be combined with or concomitantly administered with the alendronate formulation.

### Example 9

### Once-Weekly Dosing Regimen for Treatment of Paget's Disease of Bone

The alendronate-vitamin D formulation from Example 8 containing about 280 mg of alendronate in about 75 mL of liquid, on an alendronic acid active basis, may be orally administered to a human patient once-weekly, preferably about once every seven days (for example, every Sunday), for a period of at least one to six months. This method of administration is useful and convenient for treating Paget's disease of bone, particularly in humans with difficulty in swallowing tablets, while minimizing adverse upper gastrointestinal effects, particularly esophageal irritation. This method is also useful for improving patient acceptance and compliance.

### Example 10

### Liquid Formulation Containing 560 Mg of Alendronate Monosodium Trihydrate-Vitamin D

A liquid formulation containing 560 mg of alendronate monosodium trihydrate, on an alendronic acid active basis, in about 75 mL of liquid may be prepared using the following relative weights of ingredients:

| | |
|---|---|
| Alendronate monosodium trihydrate* | 9.748 mg/mL |
| Sodium Citrate | 21.18 mg/mL |
| Sodium hydroxide | qs pH 6.8 |
| Hydrochloric acid | qs pH 6.8 |
| Purified Water | qs 1.00 mL |

Additional agents such as cosolvents, flavoring agents, coloring agents, preservatives, and stabilizers may also be specifically incorporated in the formulation as follows:

| | |
|---|---|
| Alendronate monosodium trihydrate* | 9.748 mg/mL |
| Sodium Citrate | 21.18 mg/mL |
| Sodium propylparaben | 0.2250 mg/mL |
| Sodium butylparaben | 0.07500 mg/mL |
| Sodium saccharin | 0.100 mg/mL |
| Flavor | qs for taste |
| Sodium hydroxide | qs pH 6.8 |
| Hydrochloric acid | qs pH 6.8 |
| Purified Water | qs 1.00 mL |

The method of manufacture the alendronate formulation may be the same as that for Example 6.

### Example 11

### Biweekly Dosing Regimen for Treatment of Paget's Disease of Bone

The alendronate-vitamin D formulation from Example 10 containing about 560 mg of alendronate in about 75 mL of liquid, on an alendronic acid active basis, may be orally administered to a human patient biweekly, preferably about once every fourteen days (for example, on alternate Sundays), for a period of at least one to six months. This method of administration is useful and convenient for treating Paget's disease of bone, particularly in humans with difficulty in swallowing tablets, while minimizing adverse upper gastrointestinal effects, particularly esophageal irritation. This method is also useful for improving patient acceptance and compliance.

### Example 12

### Once-Weekly Dosing Regimen for Treatment of Metastatic Bone Disease

The alendronate-vitamin formulation from Example 8 containing about 280 mg of alendronate in about 75 mL of liquid, on an alendronic acid active basis, may be orally administered to a human patient once-weekly, preferably about once every seven days (for example, every Sunday). This method of administration is useful and convenient for treating or preventing metastatic bone disease in humans with lung, breast, and prostate cancer. The formulation is particularly beneficial in lung, breast, or prostate cancer patients who experience difficulty in swallowing tablets, while minimizing adverse upper gastrointestinal effects, particularly esophageal irritation. This method is also useful for improving patient acceptance and compliance.

### Example 13

### Once-Monthly Dosing Regimen for Treatment of Osteoporosis With 280 Mg

Alendronate-Vitamin D Composition The alendronate-vitamin D formulation from Example 8 containing about 280 mg of alendronate in about 75 mL of liquid, on an alendronic acid active basis, may be orally administered to a human patient once-monthly, preferably about once every 28, 30, or 31 days (for example, the 1^{st} of every month), for a period of at least six months to a year and possibly for numerous consecutive years. This method of administration is useful and convenient for treating osteoporosis, particularly in humans with difficulty in swallowing tablets, while minimizing adverse upper gastrointestinal effects, particularly esophageal irritation. The method is also useful for improving patient acceptance and compliance.

### Example 14

### Once-Monthly Dosing Regimen for Treatment of Osteoporosis With 560 mg Alendronate-Vitamin D Composition

The alendronate-vitamin D formulation from Example 10 containing about 560 mg of alendronate in about 75 mL of liquid, on an alendronic acid active basis, may be orally administered to a human patient once-monthly, preferably about once every 28, 30, or 31 days (for example, the 1st of every month), for a period of at least six months to a year and possibly for numerous consecutive years. This method of administration is useful and convenient for treating osteoporosis, particularly in humans with difficulty in swallowing tablets, while minimizing adverse upper gastrointestinal effects, particularly esophageal irritation. This method is also useful for improving patient acceptance and compliance.

### Example 15

### Once-Weekly Dosing Regiment for Treatment of Osteoporosis With 35 mg Alendronate-Vitamin D, Effervescent Composition

The alendronate-vitamin D formulation from Example 2 containing about 35 mg of alendronate, on an alendronic acid active basis, and 5,600 IU of vitamin D₃ metabolites may be prepared into a once-weekly, effervescent composition. Effervescent compositions are believed to provide increased bioavailability of bisphosphonates, i.e. alendronate, when administered to mammals.

An effervescent liquid formulation containing 35 mg of alendronate monosodium trihydrate, on an alendronic acid active basis, and non-activated metabolite of vitamin D₃ may be prepared using the following relative weights of ingredients:

| Component | Weight, mg |
|---|---|
| Alendronate sodium trihydrate* | 35 |
| (in mg alendronate acid) | |
| Citric acid, anhydrous (granular) | 1285 |
| Sodium bicarbonate (granular) | 1650 |
| Sodium carbonate, anhydrous | 140 |
| Flavoring agent (optional) | qs for taste |
| Colorant (optional) | qs for color |
| Sodium benzoate | 26 |
| Water | 9 |
| Metabolite of Vitamin D₃ | 560 |

To prepare the effervescent composition, sodium benzoate, sodium bicarbonate and alendronate sodium trihydrate are premixed to uniform blend; followed by premixing the colorant, flavoring agent, and sodium carbonate. The water may be slowly added to the citric acid and the components are mixed thoroughly to form a moist blend. To the citric acid blend may be added, in sequence, while slowly mixing, the sodium bicarbonate-containing blend, the sodium carbonate-colorant premix, and the metabolite of vitamin D₃. After forming a uniform mixture of the components of the blend, the mixture may be dried and granulated to provide the desires physical properties suitable for pressing tablets.* Optionally, other desirable components, such as sweeteners, lubricants, and binders may be added to the blend, as will become apparent to those skilled in the art. Tablets may be pressed therefrom using suitable size tooling, and the tablets may be cured, cooled and packaged in aluminum blisters or other suitable materials.

What is claimed is:

1. A pharmaceutical composition, comprising a non-activated metabolite of vitamin D₂ and/or D₃, and at least one bisphosphonate.

2. The pharmaceutical composition according to claim 1, wherein the bisphosphonate is of the formula: 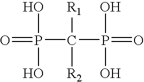
wherein R₁ is independently selected from H, OH and Cl, and R₂ is independently selected CH₃, Cl, CH₂CH₂NH₂, (CH₂)₃NH₂, CH₂-3-pyridine, CH₂—S—phenyl-Cl, CH₂CH₂N(CH₃)(pentyl), CH₂-imidazole, CH₂-2-imidazo-pyridinyl, N-(cycloheptyl), CH₂CH₂N(CH₃)₂, CH₂)₅NH₂, and CH₂-1-pyrrolidinyl, and combinations thereof.

3. The pharmaceutical composition according to claim 2, suitable for preventing or treating abnormal bone resorption, wherein the composition, comprises a pharmaceutically effective amount of at least one bisphosphonate, pharmaceutically acceptable salts, derivatives, hydrates, and mixtures thereof is from about 0.05 to about 560 mg, on an alendronic acid active weight basis, and the supplementary effective amount of non-activated metabolite of vitamin D₂ and/or D₃ is from about 100 to about 60,000 IU.

4. The pharmaceutical composition according to claim 3, wherein the bisphosphonate is selected from ibandronate, minodronate, pamidronate, risedronate, zoledronate, alendronate and combinations thereof.

5. The pharmaceutical composition according to claim 4, wherein the bisphosphonate is alendronate.

6. The pharmaceutical composition according to claim 5, suitable for administration at intervals of once-weekly, bi-weekly, monthly, twice-monthly, and bimonthly.

7. The pharmaceutical composition according to claim 6, wherein the composition is in a form selected from compressed, coated, or un-coated tablets, capsules, hard or gelatin capsules, pellets, elixirs, syrups, slurries, emulsions, suspensions, solutions, effervescent and effervescent-buffered compositions, powders, and films.

8. A pharmaceutical composition suitable for inhibiting abnormal bone resorption in a mammal, in need thereof, comprising a supplementary effective amount of from about 100 to about 60,000 IU of a non-activated metabolite of vitamin D₂ and/or D₃, and a pharmaceutically effective amount of from about 0.05 to about 560 mg of alendronate, pharmaceutically acceptable salts, derivatives, hydrates, and mixtures thereof, on an alendronic acid active weight basis.

9. A method for preventing or treating metabolic bone disease in a mammal, in need thereof, comprising concomitantly, orally administering to said mammal pharmaceutical compositions comprising a supplementary effective amount of a non-activated metabolite of vitamin D₂ and/or D₃, and a pharmaceutically effective amount of at least one bisphosphonate, as unit dosages, according to a continuous dosing schedule, wherein administration is performed, simultaneously or alternately, according to dosing intervals of once-weekly, twice-weekly, bi-weekly, once-monthly, and bi-monthly.

10. The method for preventing or treating abnormal bone resorption in a mammal according to claim 9, wherein the supplementary amount of non-activated metabolite of vitamin D₂ and/or D₃ is from about 100 to about 60,000 IU, and the pharmaceutically effective amount of alendronate is from about 0.05 to about 560 mg of alendronate, pharmaceutically acceptable salts, derivatives, hydrates, and mixtures thereof, on an alendronic acid active weight basis.

11. A method for preventing or treating abnormal bone resorption in a mammal, in need thereof, comprising orally administering to said mammal a pharmaceutical composition, comprising a supplementary effective amount of a non-activated metabolite of vitamin D₂ and/or D₃, and a pharmaceutically effective amount of at least one bisphosphonate, as a unit dosage according to a continuous administration schedule.

12. The method of treating a disease according to claim 11, wherein the disease is selected from osteoporosis, post-menopausal osteoporosis, steroid-induced osteoporosis, male osteoporosis, other disease-induced osteoporosis, idiopathic osteoporosis, and glucocorticoid-induced osteoporosis; Paget's disease; osteoarthritis, abnormally increased bone turnover; localized bone loss associated with periprosthetic bone loss or osteolysis; bone fractures; metastatic bone disease; Gaucher's disease, avascular necrosis, polyostotic fibrous dysplasia, Charcot's joint, parasitic disorders, osteogenesis imperfecta, homocystinuria, lysinuric protein intolerance, Turner's syndrome, immobilization, fibrous dysplasia, fibrogenesis imperfecta ossium, periodontal disease, tooth loss, hypercalcemia of malignancy; multiple myeloma; and osteopenia, immobilization-induced osteopenia and osteopenia due to bone metastases.

13. The method according to claim 12, wherein the bisphosphonate is of the general formula: 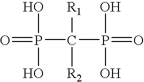
wherein R₁ is independently selected from H, OH and Cl, and R₂ is independently selected CH₃, Cl, CH₂CH₂NH₂, (CH₂)₃NH₂, CH₂-3-pyridine, CH₂—S—phenyl-Cl, CH₂CH₂N(CH₃)(pentyl), CH₂-imidazole, CH₂-2-imidazo-pyridinyl, N-(cycloheptyl), CH₂CH₂N(CH₃)₂, CH₂)₅NH₂, and CH₂-1-pyrrolidinyl, and combinations thereof.

14. The method according to claim 13, wherein the pharmaceutically effective amount of at least one bisphosphonate is from about 0.05 to about 560 mg, on an alendronic acid active weight basis.

15. The method according to claim 14, wherein the bisphosphonate is selected from ibandronate, minodronate, pamidronate, risedronate, zoledronate, alendronate and combinations thereof.

16. The method according to claim 15, wherein the bisphosphonate is alendronate.

17. The method according to claim 16, wherein the supplementary effective amount of a non-activated metabolite of vitamin D₂ and/or D₃ is from about 100 to about 60,000 IU.

18. The method according to claim 17, wherein the dosing interval is selected from once-weekly, twice-weekly, bi-weekly, once-monthly, and bi-monthly.

19. The method according to claim 18, wherein the dosing interval is once-weekly.

20. The method according to claim 19, wherein the composition is in a form selected from compressed, coated, or un-coated tablets, capsules, hard or gelatin capsules, pellets, elixirs, syrups, slurries, emulsions, suspensions, solutions, effervescent and effervescent-buffered compositions, powders, films, and the like.

21. A method for preventing or treating abnormal bone resorption in a mammal, in need thereof, comprising orally administering to said mammal a pharmaceutical composition comprising a supplementary effective amount of from about 100 to about 60,000 IU of a non-activated metabolite of vitamin D₂ and/or D₃ and a pharmaceutically effective amount of from about 0.05 to about 560 mg of alendronate, pharmaceutcally acceptable salts, derivatives, hydrates, and mixtures thereof, on an alendronic acid active weight basis, wherein the dosing interval is once-weekly, twice-weekly, bi-weekly, monthly, and bimonthly.

22. A pharmaceutical composition suitable for oral administration for the treatment or prevention of abnormal bone resorption in mammals, in need thereof, comprising a unit dosage of a supplementary effective amount of at least about 2,800 IU of a non-activated metabolite of vitamin D₂ and/or D₃, and a pharmaceutically effective amount of at least about 70 mg of alendronate, pharmaceutically acceptable salts, derivatives, hydrates, and mixtures thereof, on an alendronic acid active weight basis, wherein the dosing interval is once-weekly, twice-weekly, biweekly, monthly, and bimonthly.

23. A pharmaceutical composition suitable for oral administration for the treatment or prevention of abnormal bone resorption in mammals, in need thereof, comprising a unit dosage of a supplementary effective amount of at least about 5,600 IU of a non-activated metabolite of vitamin D₂ and/or D₃, and a pharmaceutically effective amount of at least about 70 mg of alendronate, pharmaceutically acceptable salts, derivatives, hydrates s, and mixtures thereof, on an alendronic acid active weight basis, wherein the dosing interval is once-weekly, twice-weekly, bi-weekly, monthly, and bimonthly.

24. A pharmaceutical composition suitable for oral administration for the treatment or prevention of abnormal bone resorption in mammals, in need thereof, comprising a unit dosage of a supplementary effective amount of at least about 2,800 IU of a non-activated metabolite of vitamin D₂ and/or D₃, and a pharmaceutically effective amount of at least about 35 mg of alendronate, pharmaceutically acceptable salts, derivatives, hydrates, and mixtures thereof, on an alendronic acid active weight basis, wherein the dosing interval is once-weekly, twice-weekly, bi-weekly, monthly, and bimonthly.

25. A pharmaceutical composition suitable for oral administration for the treatment or prevention of abnormal bone resorption in mammals, in need thereof, comprising a unit dosage of a supplementary effective amount of at least about 5,600 IU of a non-activated metabolite of vitamin D₂ and/or D₃, and a pharmaceutically effective amount of at least about 35 mg of alendronate, pharmaceutically acceptable salts, derivatives, hydrates, and mixtures thereof, on an alendronic acid active weight basis, wherein the dosing interval is once-weekly, twice-weekly, biweekly, monthly, and bimonthly.

26. A method of preventing or treating abnormal bone resorption in a mammal, in need thereof, comprising orally administering to said mammal a pharmaceutical composition comprising a unit dosage of a supplementary effective amount of at least about 2,800 IU of a non-activated metabolite of vitamin D₂ and/or D₃ and a pharmaceutically effective amount of at least about 70 mg of alendronate, pharmaceutically acceptable salts, derivatives, hydrates, and mixtures thereof, on an alendronic acid active weight basis, wherein dosing interval is once-weekly, twice-weekly, bi-weekly, monthly, and bimonthly.

27. A method of preventing or treating abnormal bone resorption in a mammal, in need thereof, comprising orally administering to said mammal a pharmaceutical composition comprising a unit dosage of a supplementary effective amount of at least about 5,600 IU of a non-activated metabolite of vitamin D₂ and/or D₃ and a pharmaceutically effective amount of at least about 70 mg of alendronate, pharmaceutically acceptable salts, derivatives, hydrates, and mixtures thereof, on an alendronic acid active weight basis, wherein the dosing interval is once-weekly, twice-weekly, bi-weekly, monthly, and bimonthly.

28. A method of preventing or treating abnormal bone resorption in a mammal, in need thereof, comprising orally administering to said mammal a pharmaceutical composition comprising a unit dosage of a supplementary effective amount of at least about 2,800 IU of a non-activated metabolite of vitamin D₂ and/or D₃ and a pharmaceutically effective amount of at least about 35 mg of alendronate, pharmaceutically acceptable salts, derivatives, hydrates, and mixtures thereof, on an alendronic acid active weight basis, wherein the dosing interval is once-weekly, twice-weekly, bi-weekly, monthly, and bimonthly.

29. A method of preventing or treating abnormal bone resorption in a mammal, in need thereof, comprising orally administering to said mammal a pharmaceutical composition comprising a unit dosage of a supplementary effective amount of at least about 5,600 IU of a non-activated metabolite of vitamin D₂ and/or D₃ and a pharmaceutically effective amount of at least about 35 mg of alendronate, pharmaceutically acceptable salts, derivatives, hydrates, and mixtures thereof, on an alendronic acid active weight basis, wherein the dosing interval is once-weekly, twice-weekly, bi-weekly, monthly, and bimonthly.

30. A pharmaceutical composition suitable for oral administration for the treatment or prevention of abnormal bone resorption in mammals, in need thereof, comprising a unit dosage of a supplementary effective amount of at least about 2,800 IU of a non-activated metabolite of vitamin D₂ and/or D₃, and a pharmaceutically effective amount of at least about 280 mg of alendronate, pharmaceutically acceptable salts, derivatives, hydrates, and mixtures thereof, on an alendronic acid active weight basis, wherein the dosing interval is once-weekly, twice-weekly, bi-weekly, monthly, and bimonthly.

31. A pharmaceutical composition suitable for oral administration for the treatment or prevention of abnormal bone resorption in mammals, in need thereof, comprising a unit dosage of a supplementary effective amount of at least about 5,600 IU of a non-activated metabolite of vitamin D₂ and/or D₃, and a pharmaceutically effective amount of at least about 280 mg of alendronate, pharmaceutically acceptable salts, derivatives, hydrates, and mixtures thereof, on an alendronic acid active weight basis, wherein the dosing interval is once-weekly, twice-weekly, bi-weekly, monthly, and bimonthly.

32. A method of preventing or treating abnormal bone resorption in a mammal, in need thereof, comprising orally administering to said mammal a pharmaceutical composition comprising a unit dosage of a supplementary effective amount of at least about 2,800 IU of a non-activated metabolite of vitamin D₂ and/or D₃ and a pharmaceutically effective amount of at least about 280 mg of alendronate, pharmaceutically acceptable salts, derivatives, hydrates, and mixtures thereof, on an alendronic acid active weight basis, wherein the dosing interval is once-weekly, twice-weekly, bi-weekly, monthly, and bimonthly.

33. A method of preventing or treating abnormal bone resorption in a mammal, in need thereof, comprising orally administering to said mammal a pharmaceutical composition comprising a unit dosage of a supplementary effective amount of at least about 5,600 IU of a non-activated metabolite of vitamin D₂ and/or D₃ and a pharmaceutically effective amount of at least about 35 mg of alendronate, pharmaceutically acceptable salts, derivatives, hydrates, and mixtures thereof, on an alendronic acid active weight basis, wherein the dosing interval is once-weekly, twice-weekly, bi-weekly, monthly, and bimonthly.

34. The pharmaceutical composition according to claim 27, wherein the abnormal bone resorption is selected from osteoporosis, osteopenia, Paget's disease, osteoarthritis, rheumatoid arthritis, metastatic bone disease, Gaucher's disease, avascular necrosis, polyostotic fibrous dysplasia, Charcot's joint, osteogenesis imperfecta, homocystinuria, lysinuric protein intolerance, Turner's syndrome, immobilization, fibrous dysplasia, fibrogenesis imperfecta ossium, periodontal disease, tooth loss, hypercalcemia of malignancy, and multiple myeloma.

35. The pharmaceutical composition according to claim 34, wherein the form of the composition is selected from compressed tablets, coated tablets, un-coated tablets, capsules, hard capsules, gelatin capsules, pellets, elixirs, syrups, slurries, emulsions, suspensions, solutions, effervescent, buffered-effervescent, powders, and films.
